(19) United States
(12) Patent Application Publication
(10) Pub. No.: US 2010/0055040 A1
(43) Pub. Date: Mar. 4, 2010

(54) Optical Imaging Contrast Agents

(76) Inventors: Muthunadar P. Periasamy, Chesterfield, MO (US); Brian D. Doty, St. Peters, MO (US)

Correspondence Address: Mallinckrodt Inc., 675 McDonnell Boulevard, HAZELWOOD, MO, 63042, US

(21) Appl. No.: 11/993,064

(22) PCT Filed: Jun. 21, 2006
(86) PCT No.: PCT/US06/24373

§ 371 Date: Dec. 19, 2007

Related U.S. Application Data
(60) Provisional application No. 60/693,118, filed on Jun. 21, 2005.

(51) Int. Cl. A61B 5/055; A61K 49/00

(52) U.S. Cl. 424/9.35; 424/9.1; 424/9.6

(57) ABSTRACT

The present invention provides compositions and methods for the selective identification of damaged sites in the gastrointestinal tract. More particularly, the compositions comprise a phosphated and/or sulfated saccharide, such as sucralfate, containing one or more optical molecules.

### BACKGROUND OF THE INVENTION

The present invention generally relates to compositions and methods for the selective identification of diseased or damaged sites in the gastrointestinal tract. More particularly, the present invention relates to sulfated and/or phosphated saccharides or physiologically acceptable salts thereof, more specifically polysulfated and/or polyphosphated saccharide metal salts, such as sucralfate, labeled with or containing at least one optical molecule, and the use of these sulfated and/or phosphated saccharides containing an optical molecule for the identification of ulcers in the gastrointestinal tract.

The diagnosis of gastric ulcerations and lesions generally requires the visualization of the stomach and gastrointestinal tract. A particular problem associated with the diagnosis of ulcerations and lesions involves distinguishing inflamed or ulcerated areas of the gastrointestinal lining from unulcerated, healthy sites. Current diagnostic methods may not always identify the presence of an ulcer when one exists, and may be associated with patient discomfort. Such misdiagnosis can cause gastrointestinal problems to worsen over time.

One common diagnostic method utilized to identify problem areas is endoscopy. An upper endoscopy, also called esophagogastroduodenoscopy (EGD), allows a physician to visually examine the lining of the esophagus, stomach, and upper duodenum. A thin, flexible, lighted tube, typically also comprising a small camera, is inserted through the mouth and down the throat. The endoscope transmits images of the inside of the esophagus, stomach, and duodenum, allowing the physician to see abnormalities such as inflammation or bleeding that may not otherwise be easily identified. Although endoscopy is typically more accurate than some other imaging techniques, such as x-ray imaging, endoscopy has several drawbacks, including patient discomfort due to the invasive nature of the procedure, and the necessity of using a sedative and analgesic. Patients receiving an EGD may also suffer a sore throat, and risk possible bleeding and puncture of the stomach lining.

X-rays may also be used to diagnose gastric ulcerations. Typically an x-ray contrast agent, such as a radiopaque agent, is administered orally prior to the x-ray to enhance contrast. The x-ray contrast agent is commonly a water-soluble iodinated compound. Since the iodine in these compounds blocks x-rays, the buildup or localization of the iodine in the area to be x-rayed, such as the stomach, provides the needed contrast between the stomach and other tissues for the formation of an x-ray picture. This contrast allows a physician to visualize problems and abnormalities in the stomach.

In addition to iodinated agents, barium sulfate is frequently used for x-ray examination of the gastrointestinal system. Like other x-ray contrast agents, barium sulfate may be administered orally, and coats the upper gastrointestinal tract, rendering it opaque to x-rays. The esophagus, stomach, and/or duodenum may then be evaluated.

The use of barium sulfate and other x-ray contrast agents has several drawbacks. For example, when administered orally, barium sulfate does not always coat the entire gastrointestinal mucosa, which may result in a failure to properly diagnosis gastric ulcers. Furthermore, the chalkiness of the barium sulfate may make ingestion unpalatable and unpleasant for the patient, and ingestion of the barium sulfate may cause constipation, diarrhea, or cramping.

Other methods of imaging the gastrointestinal tract and detecting ulcers involve the use of sulfated sugars, such as sucralfate, that selectively bind to ulcerated areas of the gastrointestinal tract. Some examples of gastrointestinal imaging techniques using sucralfate are described in U.S. Pat. No. 4,851,209 and U.S. Pat. No. 5,023,072, herein incorporated by reference in their entirety. U.S. Pat No. 4,851,209 describes the use of sucralfate and potassium sucrose sulfate labeled with Technetium-99m (Tc-99m) in scintigraphic imaging of the gastrointestinal area. U.S. Pat. No. 5,023,072 describes sucralfate and potassium sucrose sulfate molecules labeled with a paramagnetic, superparamagnetic, or ferromagnetic ion or particle. The labeled sucralfate may be used in magnetic resonance imaging (MRI) of the gastrointestinal area.

Another method for evaluating the gastrointestinal tract is biomedical optics, which is a relatively new technology that has a large number of potential diagnostic and therapeutic applications. With imaging, biomedical optics may have some favorable features as compared to current imaging techniques such as commuted tomography, magnetic resonance imaging, and nuclear medicine. Some benefits include: (1) the need for only a small amount of contrast agent; (2) the use of a non-radioactive energy source; and (3) the use of relatively inexpensive instruments.

Although there are a number of imaging agents and methods available for imaging and analyzing the gastrointestinal tract, there continues to be a need for imaging agents and methods that can provide quick, accurate evaluation without discomfort to the patient. It would be beneficial if the imaging agent could provide high contrast and specific targeting to ulcerated areas of the gastrointestinal tract.

### SUMMARY OF THE INVENTION

The present invention relates to compositions and methods for the selective identification of diseased or damaged sites in the gastrointestinal tract. More particularly, the present invention relates to sulfated and/or phosphated saccharides or physiologically acceptable salts thereof, more specifically polysulfated and/or polyphosphated saccharide metal salts, such as sucralfate, comprising at least one optical molecule, and the use of the sulfated and/or phosphated saccharides or physiologically acceptable salts thereof comprising at least one optical molecule for the identification of gastric ulcers in the gastrointestinal tract. The ability of the optical molecule-containing saccharide class of compounds and derivatives thereof to selectively bind to gastric ulcers and lesions in the gastrointestinal tract enables visualization of the ulcers and lesions using a variety of optical detection techniques.

### DETAILED DESCRIPTION OF THE INVENTION

In accordance with the present invention, it has been discovered that certain saccharide molecules that are known to selectively bind to ulcerated and/or inflamed sites of the gastrointestinal tract upon introduction therein may be substituted with at least one optical molecule to produce an optical imaging agent that can be used to specifically target and image inflamed or ulcerated sites of the gastrointestinal tract. As used herein, “optical molecule(s)” means optical dyes, nanoparticles, and optical dye encapsulated in a physiologically acceptable particulate material that are suitable for optical detection in the gastrointestinal tract. Although discussed primarily herein as a suitable optical dye chemically introduced into the sulfated and/or phosphated saccharides, it should be understood that suitable nanoparticles (including quantum dots), or optical dyes encapsulated in a physiologically acceptable particulate material, which are physically introduced into the sulfated and/or phosphated saccharides and which can be optically detected in the gastrointestinal tract can be used in accordance with the present invention to produce a suitable optical imaging contrast agent. As used herein, “substituted” means the chemical introduction of a compound or molecule onto a saccharide molecule. As used herein with respect to saccharides of the invention, the terms “molecule(s)” and “compound(s)” are used interchangeably. Specific sites on the saccharide compounds where substitution may occur are discussed below. By chemically or physically introducing an optical molecule into the saccharide compound, it is possible to accurately identify diseased or damaged sites of the gastrointestinal tract using compositions and methods that are more reliable, less invasive, and safer than other compositions and methods currently in use.

The optical imaging agents of the present invention comprise saccharide molecules containing at least one optical molecule, e.g., substituted with at least one optical dye, capable of being optically detected in the gastrointestinal tract. The saccharide molecule may be any physiologically acceptable substituted saccharide, or physiologically acceptable salt thereof, that selectively binds to ulcerated and/or inflamed sites of the gastrointestinal tract. Preferably the saccharide molecules are sulfated and/or phosphated saccharides or physiologically acceptable salts thereof. As used herein, the terms “sulfated saccharide” or “saccharide sulfate,” used interchangeably, refer to a saccharide molecule comprising one or more sulfate groups. Similarly, the terms “phosphated saccharide” or “saccharide phosphate,” used interchangeably, refer to a saccharide molecule comprising one or more phosphate groups. Preferably, the sulfated and/or phosphated saccharide is polysulfated and/or polyphosphated, i.e., has two or more sulfate and/or phosphate groups.

The saccharide itself that may be used in the optical imaging agents of the present invention includes monosaccharides and polysaccharides. As used herein, the term polysaccharides is meant to encompass disaccharides as well as tri-, tetra-, and oligosaccharides. Examples of suitable saccharides include fructose, glucose, ribose, mannose, lactose, maltose, sucrose, erythrose, threose, arabinose, deoxyribose, cellobiose, trehalose, melezitose, and stachyose, among others, or any combination thereof. Preferably, the saccharide is a disaccharide. The currently preferred disaccharides are sucrose, lactose, or maltose. More preferably, the disaccharide is sucrose. The optical imaging compositions of the present invention may thus include substituted monosaccharides, substituted polysaccharides, or combinations thereof. Any substituted saccharide that selectively binds to damaged and/or inflamed sites of the gastrointestinal tract may be suitable for use in the present invention.

The saccharides of the present invention may thus comprise: (1) mono- or poly-sulfated saccharides; (2) mono- or poly-phosphated saccharides; (3) saccharides that have been both phosphated and sulfated (i.e., sulfated-phosphated saccharides), (4) any other physiologically acceptable substituted saccharide that selectively binds to ulcerated and/or inflamed sites of the gastrointestinal tract; and (5) mixtures thereof. In general, the sulfated and/or phosphated saccharide molecules used herein will bind with greater affinity to damaged sites of the gastrointestinal tract, such as inflamed and ulcerated sites, than to healthy sites. This affinity allows the optical molecules chemically or physically introduced into the sulfated and/or phosphated saccharide molecules to be specifically targeted to damaged sites in the gastrointestinal tract, thus enhancing ulcer detection during examination.

Advantageously, physiologically acceptable salts of the saccharides may be used herein. Physiologically acceptable salts include salts formed with suitable bases. Examples of such suitable salts are salts of alkali metals (e.g., sodium and potassium), alkaline earth metals (e.g., calcium, magnesium and barium), aluminum, ammonium (NH₄⁺), and combinations thereof. For example, saccharide metal salts such as aluminum, calcium, magnesium, sodium, potassium, barium, or any other physiologically acceptable salt, and combinations thereof may be used. In one embodiment, the salt is aluminum salt.

In another embodiment, the saccharide is a polysulfated disaccharide aluminum salt. One example of such a saccharide is sucralfate (α-D-glucopyranoside, β-D-fructofuranosyl-, octakis-(hydrogen sulfate), aluminum complex). Sucralfate (commercially available as CARAFATE®, Hoechst Marion Roussel) is a basic aluminum complex of sulfated sucrose, having general formula (1):

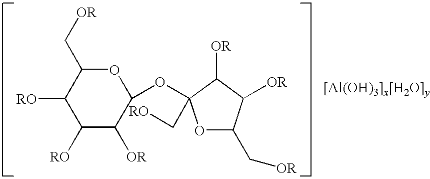

wherein R is —SO₃Al(OH)₂; x is 8 to 10; and y is 22 to 31.

Sucralfate is used in the treatment of peptic, duodenal, and prepyloric ulcers, gastritis, reflux esophagitis, and other gastrointestinal irritations. Although the precise mechanism of sucralfate's ability to accelerate healing of gastrointestinal problems is yet to be fully defined, it is known that sucralfate binds selectively to ulcerated, rather than unulcerated, areas in the gastrointestinal tract. Specifically, it has been shown that approximately six to seven times more sucralfate binds to ulcerated gastric mucosa than to unulcerated mucosa in humans.

Sucralfate coats gastric and peptic ulcerated tissue by adhering to the proteinaceous exudate at the ulcer site and forming a protective barrier to acid, pepsin, or bile salts. It is believed that this protective barrier facilitates healing of the ulcerated gastric tissue by blocking the damaged site from further attack. Sucralfate may also exert an anti-ulcer effect by depletion of acid, pepsin, and bile salts from the gastric secretion, and by promoting revascularization and regeneration of ulcerated mucosal tissue.

The present invention combines a sulfated and/or phosphated saccharide with a suitable optical molecule to produce an optical imaging agent that can be used to specifically target and image inflamed or ulcerated sites of the gastrointestinal tract. Although discussed primarily in terms of sucralfate and related structures, it should be understood that any saccharide composition that has binding specificity for gastrointestinal ulcerated mucosa or other gastrointestinal diseases can be chemically modified in accordance with the present invention to produce a suitable optical imaging contrast agent. Other suitable compounds may include, in addition to those discussed above, for example, other physiologically acceptable polysulfated and/or polyphosphated saccharide metal salts, and derivatives of sucralfate, among others.

Suitable dyes for substitution into or attachment onto a sulfated and/or phosphated saccharide molecule are optical dyes. As used herein, the term “optical dye” means a dye that transmits, reflects, or fluoresces light in the wavelength range of about 350 nanometers to about 1300 nanometers, and is sufficiently stable under the conditions found in the gastrointestinal tract, e.g., in aqueous systems under acidic conditions. As used herein, “sufficiently stable” means stable for a time sufficient to permit administration of the optical imaging agent of the present invention to the patient and obtaining the optical image. For example, for imaging in the upper gastrointestinal tract, the sufficiently stable optical dyes are stable under the pH conditions in the upper gastrointestinal tract (e.g., stable at pH <5, preferably pH <3) for up to about 30 minutes to 4 hours, more particularly up to about 30 minutes to 2 hours. The optical dyes of the present invention preferably do not contain aliphatic chains or groups substituted with primary amine, secondary amine, aldehyde or ketone groups. The optical dyes described herein and attached to a sulfated and/or phosphated saccharide molecule allow ulcerated and inflamed sites of the gastrointestinal tract to be detected using various optical imaging techniques, such as, for example, various optical tomographic, endoscopic, photoacoustic, and sonofluorescence applications.

Any suitable optical dye, or derivative thereof, known in the art may be used in combination with sulfated and/or phosphated saccharide in accordance with the present invention. In one embodiment, suitable optical dye molecules in accordance with the present invention comprise at least one substituent selected from —SO₃H (sulfonic acid), —PO₄H₂ (phosphoric acid), —COOH (carboxylic acid), and combinations thereof. In a preferred embodiment, the optical dye molecule comprises at least two of the acid substituents. If the optical dye molecule comprises at least two acid substituents, at least two of the acid substituents are optionally in close proximity to one another in the optical dye structure. It is believed that the acid substituents being in close proximity to one another may enable stable salt formation with divalent or trivalent metal ions. The optical dye molecules of the present invention are believed to have low toxicity. Examples of optical dye molecules containing two or more sulfonic acid groups or their salts are described in U.S. Pat. No. 6,329,531, herein incorporated by reference in its entirety. Examples of optical dye molecules containing three or more sulfonic acid groups are described in WO 01/43781, herein incorporated by reference in its entirety. Food dyes containing one or more —SO₃H or —PO₄H₂ groups and meeting the other requirement of optical dyes according to the present invention may also be suitable for use in the present invention.

For example, suitable optical dyes include: cyanines, indocyanines (such as indocyanine green), phthalocyanines, squaraines, polymethines, pyrazines, rhodamines, fluoresceins, and derivatives and analogues thereof.

Examples of these and other suitable optical dyes are known in the art. For example, suitable cyanine and/or indocyanine dyes are described in U.S. Pat. No. 6,180,085; U.S. Pat. No. 6,180,087; U.S. Pat. No. 5,453,505; U.S. Pat No. 6,258,340; U.S. Pat App. Pub. No. 2003/0180221; WO 00/16810; and WO 01/43781, all herein incorporated by reference in their entirety. Other suitable optical dyes are described in U.S. Pat. No. 6,277,841; and U.S. Pat. No. 6,540,981, all herein incorporated by reference in their entirety. Based on the description of the present invention, it will be readily apparent to one skilled in the art which of the dyes disclosed in each of the above-listed patents and published patent applications will be suitable optical dyes of the present invention.

Preparation of the optical dyes of the present invention can be done using conventional processes well known to those of ordinary skill in the art. For example, see the above patents and published applications, such as WO 01/43781, for descriptions of how to prepare the optical dyes taught therein.

In one embodiment, the optical dye for incorporation into the sulfated and/or phosphated saccharide is a cyanine dye, indocyanine dye, or a derivative thereof, such as those described in U.S. Pat. Nos. 6,180,087 and 6,180,085. In one embodiment, the cyanine dye is a dye of general formula (2):

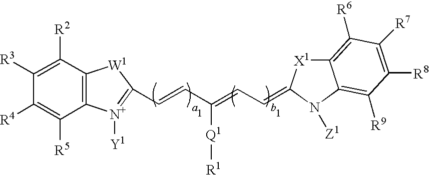

wherein a₁ and b₁ vary from 0 to 5; W¹ and X¹ may be the same or different and are selected from the group consisting of —CR¹⁰R¹¹, —O—, —NR¹², —S—, and —Se; Q¹ is a single bond or is selected from the group consisting of —O—, —S—, —Se—, and —NR¹³; Y¹ and Z¹ may be the same or different and are selected from the group consisting of —(CH₂)_{c}—CO₂H, —(CH₂)_{c}—SO₃H, —(CH₂)_{c}—PO₄H₂, —CH₂—(CH₂—O—CH₂)_{d}—CH₂—CO₂H, —CH₂—(CH₂—O—CH₂)_{d}—CH₂—SO₃H, —CH₂—(CH₂—O—CH₂)_{d}—CH₂—PO₄H₂, —CH₂)_{g}—N(R¹⁴)—(CH₂)ₕ—CO₂H, —(CH₂)_{g}—N(R¹⁴)—(CH₂)ₕ—SO₃H, —(CH₂)_{g}—N(R¹⁴)—(CH₂)ₕ—PO₄H₂, —(CH₂)ᵢ—N(R¹⁵)—CH₂—(CH₂—O—CH₂)ⱼ—CH₂—CO₂H, —(CH₂)ᵢ—N(R¹⁵)—CH₂—(CH₂—O—CH₂)ⱼ—CH₂—SO₃H; and —(CH₂)ᵢ—N(R¹⁵)—CH₂—(CH₂—O—CH₂)ⱼ—CH₂—PO₄H₂; R¹ and R¹⁰ to R¹⁵ may be the same or different and are selected from the group consisting of hydrogen, C1-C10 alkyl, C1-C10 aryl, C1-C10 alkoxyl, C1-C10 polyalkoxyalkyl, —CH₂(CH₂—O—CH₂)_{c}—CH₂—OH, C1-C20 polyhydroxyalkyl, C1-C10 polyhydroxyaryl, —(CH₂)_{d}—CO₂H, —(CH₂)_{d}—SO₃H, —(CH₂)_{d}—PO₄H₂, —CH₂—(CH₂—O—CH₂)ₑ—CH₂—CO₂H, —CH₂—(CH₂—O—CH₂)ₑ—CH₂—SO₃H, and —CH₂—(CH₂—O—CH₂)ₑ—CH₂—PO₄H₂; c, e, g, h, and i vary from 1 to 10; d, f, and j vary from 1 to 100; and R² to R⁹ may be the same or different and are selected from the group consisting of hydrogen, C1-C10 alkyl, C1-C10 aryl, hydroxyl, C1-C10 polyhydroxyalkyl, C1-C10 alkoxyl, cyano, nitro, —COOH, —SO₃H, —PO₄H₂, and halogen.

In another embodiment, the cyanine dye is a dye of general formula (3):

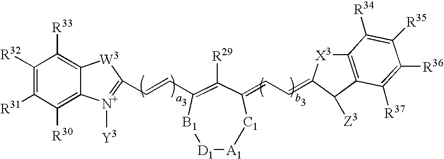

wherein a₃ and b₃ are defined in the same manner as a₁, and b₁; W³ and X³ are defined in the same manner as W¹ and X¹; Y³ is defined in the same manner as Y¹; Z³ is defined in the same manner as Z¹; A₁ is a single or a double bond; if A₁ is a single bond, then B₁ and C₁ may be the same or different and are selected from the group consisting of —O—, —S—, —Se—, —P—, and —NR³⁸ and D₁ is selected from the group consisting of —CR³⁹R⁴⁰, and —C═O; if A₁ is a double bond, then B₁ is selected from the group consisting of —O—, —S—, —Se—, —P—, and —NR³⁸, C₁ is nitrogen or —CR⁴¹, and D₁ is —CR⁴²; R²⁹ to R³⁷ are selected from the group consisting of hydrogen, C1-C10 alkyl, C1-C10 aryl, hydroxyl, hydrophilic peptide, C1-C10 polyhydroxyalkyl, C1-C10 alkoxyl, —COOH, —SO₃H, —PO₄H₂, cyano, nitro, and halogen; R³⁸ to R⁴² may be the same or different and are selected from the group consisting of hydrogen, C1-C10 alkyl, C1-C10 aryl, C1-C10 alkoxyl, C1-C10 polyalkoxyalkyl, —CH₂(CH₂—O—CH₂)_{c}—CH₂—OH, C1-C20 polyhydroxyalkyl, C1-C10 polyhydroxyaryl, —CH₂)_{d}—CO₂H, —(CH₂)_{d}—SO₃H, —CH₂)_{d}—PO₄H₂, —CH₂—(CH₂—O—CH₂)ₑ—CH₂—CO₂H, —CH₂—(CH₂—O—CH₂)ₑ—CH₂—SO₃H, and —CH₂—(CH₂—O—CH₂)_{c}—CH₂—PO₄H₂; c, e, g, h, and i vary from 1 to 10; d, f, and j vary from 1 to 100; R⁴³ and R⁴⁴ may be the same or different and are selected from the group consisting of hydrogen, C1-C10 alkyl, and C1-C10 aryl, or together form a 5, 6, or 7 membered heterocyclic ring optionally containing one or more oxygen, nitrogen, or a sulfur atom.

In yet another embodiment, the cyanine dye is a dye of general formula (4):

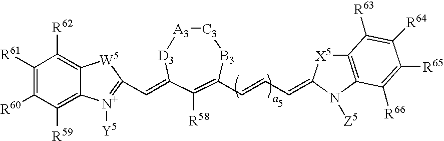

wherein a₅ is defined in the same manner as a₁; W⁵ and X⁵ are defined in the same manner as W¹ and X¹; Y⁵ is defined in the same manner as Y¹; Z⁵ is defined in the same manner as Z¹; A₃ is defined in the same manner as A₁; B₃, C₃, and D₃ are defined in the same manner as B₁, C₁, and D₁; and R⁵⁸ to R⁶⁶ are defined in the same manner as R²⁹ to R³⁷.

The optical dye used herein may also be an indocyanine dye, for example, indocyanine green. The indocyanine dye may be an indocyanine dye such as those described in U.S. Pat. Nos. 6,180,087 and 6,180,085. For example, in one embodiment, the indocyanine dye is a dye of general formula (5):

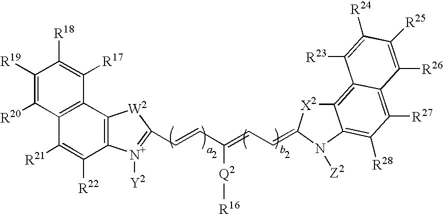

wherein a₂ and b₂ are defined in the same manner as a₁, and b₁; W² and X² are defined in the same manner as W¹ and X¹; Q² is defined in the same manner as Q¹; R¹⁶ and R¹⁰ to R¹⁵ are defined in the same manner as R¹ and R¹⁰ to R¹⁵; Y² is defined in the same manner as Y¹; Z² is defined in the same manner as Z¹; and R¹⁷ to R¹⁸ are defined in the same manner as R² to R⁹.

In one specific embodiment, the sulfated and/or phosphated saccharide molecules described herein may be substituted with more than one optical dye. Because of the specificity of the sulfated and/or phosphated saccharide molecule (such as sucralfate and its precursors and derivatives) for damaged areas of the gastrointestinal tract, only a small amount of optical dye is required for detection. This is particularly advantageous because the use of small amounts of dye reduces the risk of dye toxicity to the patient.

The phrase “optical dye substituted saccharide sulfate composition” or “optical dye substituted sulfated saccharide composition”, used interchangeably herein, refers to a composition comprising at least one sulfated saccharide molecule that is substituted at one or more position on the sulfated saccharide with at least one optical dye. Likewise, the phrase “optical dye substituted saccharide phosphate composition” or “optical dye substituted phosphated saccharide composition”, used interchangeably herein, refers to a composition comprising at least one phosphated saccharide molecule that is substituted at one or more position on the phosphated saccharide with at least one optical dye. Not every sulfated and/or phosphated saccharide molecule in the composition needs to be substituted with an optical dye, so long as a sufficient amount of the sulfated and/or phosphated saccharide molecules are substituted to enable detection of damaged areas of the gastrointestinal tract.

The amount of optical molecule, and particularly optical dye, required for detection will vary depending on the optical molecule selected and optical detection technique used, and may readily be determined by one skilled in the art. For example, the amount of optical dye in an optical dye substituted sulfated and/or phosphated saccharide composition may be specified as the mole ratio of total optical dye to total sulfated and/or phosphated saccharide and may be in the ranges of about 0.0001:1 to about 3:1, 0.001:1 to about 1:1, about 0.002:1 to about 1:1, and about 0.005:1 to about 0.5:1. For example, 0.01 mole of optical dye may be reacted with 1 mole of sucralfate to produce a composition comprising optical dye substituted sucralfate with a mole ratio of optical dye to sucralfate of about 0.01:1. The composition of the present invention may comprise sulfated and/or phosphated saccharide molecules substituted at one position with the optical dye, sulfated and/or phosphated saccharide molecules substituted at multiple positions with the optical dye, unsubstituted sulfated and/or phosphated saccharide molecules, and combinations thereof.

The optical dye substituted sulfated and/or phosphated saccharide compositions of the present invention may optionally comprise more than one type of optical dye. Such compositions may advantageously enable the detection of damaged areas of the gastrointestinal tract using multiple detection methods. In one embodiment, the optical dyes are selected from the group consisting of cyanine dyes and indocyanine dyes.

For example, in one embodiment, an optical dye substituted sulfated and/or phosphated saccharide composition comprising two or more types of optical dye may be prepared by reacting two or more types of optical dye with the sulfated and/or phosphated saccharide. The resulting composition may comprise sulfated and/or phosphated saccharide molecules substituted with only one of the optical dyes, sulfated and/or phosphated saccharide molecules substituted with two or more of the optical dyes, unsubstituted sulfated and/or phosphated saccharide molecules, and combinations thereof. Alternatively, an optical dye substituted sulfated and/or phosphated saccharide composition comprising two or more types of optical dye may be prepared by separately reacting each optical dye with sulfated and/or phosphated saccharide molecules to produce optical dye substituted sulfated and/or phosphated saccharide compositions comprising each dye, and combining the resulting compositions to produce an optical dye substituted sulfated and/or phosphated saccharide composition comprising two or more optical dyes. In one embodiment, the optical dye substituted sulfated and/or phosphated saccharide composition comprises a mole ratio of total optical dye to total sulfated and/or phosphated saccharide within the ranges of about 0.0001:1 to about 3:1, 0.001:1 to about 1:1, about 0.002:1 to about 1:1, and about 0.005:1 to about 0.5:1.

The optical dyes described above may be introduced or substituted into the sulfated and/or phosphated saccharide molecule at one or more positions on the molecule in accordance with the present invention. In one embodiment the optical dye is introduced into a saccharide having general formula (6):

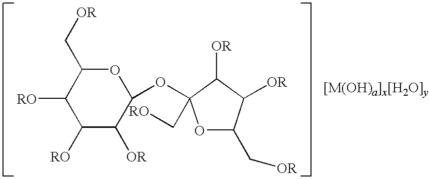

wherein R is selected from the group consisting of —SO₃M(OH)_{b}, —PO₃M(OH)_{b}, hydrogen, and any combination thereof, so long as at least one —R group is —SO₃M(OH)_{b}, or —PO₃M(OH)_{b}; M is independently selected from the group consisting of Al, Ca, Mg, Na, K, Ba and NH₄; a is the valence of M (e.g., if M is Al, a=3; if M is Mg, a=2; if M is Na, a=1, etc.; a is 1 to 3), b is the valence of M minus 1 (b is 0 to 2), x is 1 to 15; and y is 0 to 35. Thus, in one molecule, all the R groups may be —SO₃M(OH)_{b}, all the R groups may be —PO₃M(OH)_{b}, or the R groups may be some combination of —SO₃M(OH)_{b} and/or —PO₃M(OH)_{b} and/or hydrogen, so long as at least one R group is —SO₃M(OH)_{b}, or —PO₃M(OH)_{b}.

The optical dye may be substituted into the saccharide at various positions, as illustrated below. It is noted that formula 6 only encompasses sucrose-based sulfated and/or phosphated disaccharides. However, as previously discussed, the sulfated and/or phosphated saccharide may be any saccharide that selectively binds to damaged and/or inflamed areas of the gastrointestinal tract, and may include phosphated and/or sulfated monosaccharides and other polysaccharides. Consequently, the following discussion of optical dye substitution is intended to be exemplary, and not limiting, as similar substitutions may be made on other suitable saccharides.

In one embodiment, the optical dye is substituted into the saccharide molecule at any one or more of the eight —OR positions; that is, at least one (and up to all eight) of the —OR groups is eliminated and replaced with an optical dye, such that at least one optical dye replaces an —OR group and is bound, either directly or indirectly, to the ring structure or to a carbon atom that is bound to the ring structure. The optical dye substituted saccharide resulting from this type of substitution may have general formula (7):

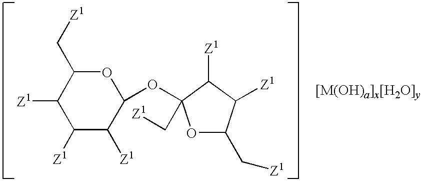

wherein Z¹ is selected from the group consisting of an optical dye, —OR, and any combination thereof, so long as at least one Z¹ group is an optical dye; R is selected from the group consisting of —SO₃M(OH)_{b}, —PO₃M(OH)_{b}, hydrogen, and any combination thereof, so long as at least one —R group is —SO₃M(OH)_{b} or —PO₃M(OH)_{b}; M is independently selected from the group consisting of Al, Ca, Mg, Na, K, Ba and NH₄; a is the valence of M (e.g., if M is Al, a=3; if M is Mg, a=2; if M is Na, a=1, etc.; a is 1 to 3), b is the valence of M minus 1 (b is 0 to 2), x is 1 to 15; and y is 0 to 35. Thus, in one molecule, the Z¹ groups may be any combination of optical dye, and/or —OR groups, so long as at least one Z¹ group is an optical dye and at least one Z¹ group is —OR. Likewise, in one molecule, all the R groups may be —SO₃M(OH)_{b}, all the R groups may be —PO₃M(OH)_{b}, or the R groups may be any combination of —SO₃M(OH)_{b}, and/or —PO₃M(OH)_{b}, and/or hydrogen, so long as at least one —R group is —SO₃M(OH)_{b} or —PO₃M(OH)_{b}.

In another embodiment, the optical dye is substituted into the saccharide molecule at any one or more of the eight —R positions; that is, at least one (and up to all eight) of the —R groups is eliminated and replaced with an optical dye, such that at least one hydroxyl oxygen will be bonded, either directly or indirectly, to the optical dye. The optical dye substituted saccharide molecule resulting from this type of substitution may have general formula (8):

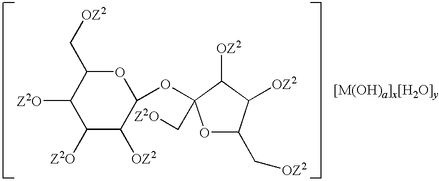

wherein Z² is selected from the group consisting of an optical dye, —R, hydrogen, and any combination thereof, so long as at least one Z² group is an optical dye, and at least one Z² group is —R; R is independently selected from —SO₃M(OH)_{b}, and —PO₃M(OH)_{b}; M is independently selected from the group consisting of Al, Ca, Mg, Na, K, Ba, and NH₄; a is the valence of M (e.g., if M is Al, a=3; if M is Mg, a=2; if M is Na, a=1, etc.; a is 1 to 3), b is the valence of M minus 1 (b is 0 to 2), x is 1 to 15; and y is 0 to 35. Thus, in one molecule, the Z² groups may be some combination of optical dye, and/or —R groups, and/or hydrogen, so long as at least one Z² group is an optical dye, and at least one Z² group is —R. By “R is independently selected from —SO₃M(OH)_{b} and —PO₃M(OH)_{b}” it is meant that in one molecule, all the R groups may be —SO₃M(OH)_{b}, all the R groups may be —PO₃M(OH)_{b}, or the R groups may be some combination of —SO₃M(OH)_{b} and —PO₃M(OH)_{b}.

In yet another embodiment, the optical dye is bonded to one or more of the metal atoms present in the saccharide molecule. In this embodiment, the optical dye is bonded either directly or indirectly to a metal atom located in the —R group, or to a metal atom located in the complexed group. The optical dye substituted saccharide resulting from this type of substitution may have general formula (9):

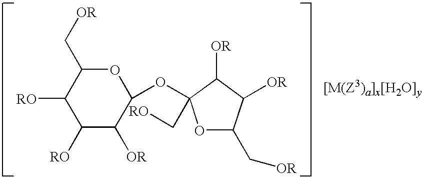

wherein a is the valence of M (e.g., if M is Al, a=3; if M is Mg, a=2; if M is Na, a=1, etc.; a is 1 to 3), b is the valence of M minus 1 (b is 0 to 2), x is 1 to 15; and y is 0 to 35; R is selected from the group consisting of —SO₃M(Z³)_{b}, —PO₃M(Z³)_{b}, hydrogen, and any combination thereof, so long as at least one —R group is —SO₃M(Z³)_{b} or —PO₃M(Z³)_{b}; M is independently selected from the group consisting of Al, Ca, Mg, Na, K, Ba and NH₄; and Z³ is selected from the group consisting of an optical dye, —OH, and any combination thereof, so long as at least one Z³ group is an optical dye. Thus, in one molecule, all the Z³ groups may be optical dye, or the Z³ groups may be some combination of optical dye, and/or —OH groups, so long as at least one Z³ group is an optical dye. For example, the optical dye may be bonded to a metal atom (such as Al, Ca, Mg, Na, K, or Ba) located in an R group (i.e., —SO₃M(Z³)_{b} and/or —PO₃M(Z³)_{b}), wherein both Z³ groups are —OH groups, both Z³ groups are optical dye, or the Z³ groups are some combination of optical dye, and/or —OH. Likewise, the optical dye may be bonded to a metal atom (such as Al, Ca, Mg, Na, K, or Ba) located in the complex group (i.e., [M(Z³)ₐ]ₓ[H₂O]_{y}), wherein all Z³ groups are optical dye, all Z³ groups are —OH groups, or the Z³ groups are some combination of optical dye, and/or —OH. However, in one molecule, at least one Z³ group should be an optical dye. In addition, in one molecule, all the R groups may be —SO₃M(Z³)_{b}, all the R groups may be —PO₃M(Z³)_{b}, or the R groups may be some combination of —SO₃M(Z³)_{b}, and/or —PO₃M(Z³)_{b}, and/or hydrogen, so long as at least one R group is —SO₃M(Z³)_{b} or —PO₃M(Z³)_{b}.

In still another embodiment, the optical dye is bonded to one or more of the hydroxyl oxygen atoms present in the saccharide molecule. In this embodiment, the optical dye is bonded either directly or indirectly to a hydroxyl oxygen atom that is also bound to a metal atom located in the —R group, or to a hydroxyl oxygen atom that is also bound to a metal atom located in the complexed group. The optical dye substituted saccharide resulting from this type of substitution may have general formula (10):

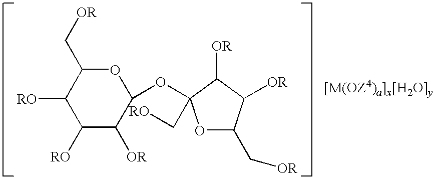

wherein R is selected from the group consisting of —SO₃M(OZ⁴)_{b}, —PO₃M(OZ⁴)_{b}, hydrogen, and any combination thereof, so long as at least one —R group is —SO₃M(Z⁴)_{b} or —PO₃M(Z⁴)_{b}; M is independently selected from the group consisting of Al, Ca, Mg, Na, K, Ba and NH₄; Z⁴ is an optical dye, hydrogen, and any combination thereof, so long as at least one Z⁴ group is an optical dye; a is the valence of M (e.g., if M is Al, a=3; if M is Mg, a=2; if M is Na, a=1, etc.; a is 1 to 3), b is the valence of M minus 1 (b is 0 to 2), x is 1 to 15; and y is 0 to 35. Thus, in one molecule, all the Z⁴ groups may be optical dye, or the Z⁴ groups may be some combination of optical dye, and/or hydrogen, so long as at least one Z⁴ group is an optical dye. For example, the optical dye may be bonded to a hydroxyl oxygen atom that is also bound to a metal atom (such as Al, Ca, Mg, Na, K, or Ba) located in the —R group (i.e., —SO₃M(OZ⁴)_{b} and/or —PO₃M(OZ⁴)_{b};), wherein both Z⁴ groups are hydrogen, both Z⁴ groups are optical dye, or the Z⁴ groups are some combination of optical dye, and/or hydrogen. Likewise, the optical dye may be bonded to a hydroxyl oxygen atom that is also bound to a metal atom (such as Al, Ca, Mg, Na, K, or Ba) located in the complex group (i.e., [M(OZ⁴)ₐ]ₓ[H₂O]_{y}), wherein all Z⁴ groups are optical dye, all Z⁴ groups are hydrogen, or the Z⁴ groups are some combination of optical dye, and/or hydrogen. However, in one molecule, at least one of the Z⁴ groups should be optical dye. In addition, in one molecule all the R groups may be —SO₃M(OZ⁴)_{b}, all the R groups may be —PO₃M(OZ⁴)_{b}, or the R groups may be some combination of —SO₃M(OZ⁴)_{b}, and/or —PO₃M(OZ⁴)_{b}, and/or hydrogen, so long as at least one —R group is —SO₃M(OZ⁴)_{b} or —PO₃M(OZ⁴)_{b}.

In another embodiment, the optical dye can be introduced into the saccharide molecule at a combination of one or more of the sites described above. For example, the optical dye can be introduced by replacing an —R group and an —OR group and bonded to a metal atom and a hydroxyl oxygen atom, or any combination thereof

As previously mentioned, one type of optical dye may be attached at multiple positions on the saccharide. Alternately, two or more different types of optical dye may be attached to the same saccharide molecule at any of the above described positions. The number of optical dyes is not critical, so long as the optical dye substituted sulfated and/or phosphated saccharide composition comprises sufficient optical dye to enable detection of damaged areas of the gastrointestinal tract.

The optical dyes described herein may be attached to the sulfated and/or phosphated saccharide molecules at one or more of the positions described above using any suitable means known in the art. For example, the optical dyes may be directly attached to the sulfated and/or phosphated saccharide molecules at the positions described above, or indirectly attached at these positions using linking means, chelator means, coupling means, and/or cross-linking means, among others. By “direct attachment” it is meant that the optical dye is not attached to the sulfated and/or phosphated saccharide by some type of connector, such as a linker, chelator, or coupler, among others. For example, the optical dye may be attached to the saccharide via one of the following or similar linkages, e.g., carbon-carbon bond, carbon-nitrogen bond, carbon-oxygen bond, oxygen-carbon bond, or oxygen-sulfur bond using conventional chemical reaction technology well known to those of ordinary skill in the art. By “indirect attachment” it is meant that the optical dye is attached to the sulfated and/or phosphated saccharide molecule by means of some type of connector. For example, suitable connector means include chelators, linkers, couplers, among others, or various combinations thereof. Because various attachment means are readily known in the art, they will be described only briefly herein.

One suitable method for directly attaching the optical dye to the sulfated and/or phosphated saccharide includes, for example, reacting the —COOH group (or its equivalent) of the dye with an —OH group of the saccharide molecule leading to the formation of an ester linkage. Another option would be to react the —OH group of an optical dye with the —OH group of the saccharide molecule resulting in the formation of an ether linkage. These and other similar methods of synthesis are well known in the art.

In another embodiment, the optical dye is attached to the sulfated and/or phosphated saccharide molecule via a linker by first attaching a functional group to the appropriate location in the sulfated and/or phosphated saccharide molecule and/or to an appropriate location in the optical dye to facilitate reaction between the sulfated and/or phosphated saccharide and optical dye. Suitable functional groups include, but are not limited to, isocyanate groups, amino groups, haloacetyl groups, tosylate groups (—OTs), and sulfonyl halides, among others. Suitable functional groups are also described in U.S. Pat No. 6,521,209. Suitable linkers include polymers and coupling agents, among others. In one embodiment, the linker is a polymer. The polymer may be initially attached to the sulfated and/or phosphated saccharide molecule and then, in a second reaction, used to connect the sulfated and/or phosphated saccharide to the optical dye. Alternatively, the polymer may be initially attached to the optical dye, and then, in a second reaction, used to connect the optical dye to the sulfated and/or phosphated saccharide molecule. In yet another alternative, the optical dye and sulfated and/or phosphated saccharide may simultaneously be attached to the polymer in one reaction. Optionally, functional groups may be added to the optical dye and/or sulfated and/or phosphated saccharide molecule to facilitate attachment to the linker.

In another embodiment, the optical molecules of the present invention may include any physiologically acceptable particulate materials containing one or more optical molecule. Such particles may be solid particles (e.g., uncoated or coated to provide stability in aqueous systems), or fluid (e.g., liquid particles in an emulsion), or may be aggregates (e.g., fluid containing liposomes) containing one or more optical molecule. In one embodiment, the particulate material has a particle size smaller than or similar to the incident light wavelength. The particles are preferably water-insoluble or at least sufficiently poorly soluble so as to retain their desired particle size for a sufficient time following administration to the patient to be optically imaged. Examples of such particulate materials are described in U.S. Pat. No. 6,540,981.

In one embodiment, these particles are nanoparticles suitable for optical imaging. Such nanoparticles can be either quantum dots, nanoparticles of Si or Si/Ge, or nanoparticles having a suitable optical dye, e.g. a fluorophore, encapsulated therein. The luminescence wavelength of nanoparticles such as quantum dots and Si or Si/Ge nanoparticles is dependent on the size of the nanoparticles. The methods for producing such nanoparticles are well known in the art. For example, U.S. Pat. No. 6,585,947, herein incorporated by reference in its entirety, discloses a method for producing uniform (1-3 nanometer in diameter) Si nanoparticles. Such nanoparticles can be physically entrapped within or coated by the sulfated and/or phosphated saccharides of the present invention, particularly the sulfated and/or phosphated saccharide metal salt matrix, and used for the optical imaging applications of the present invention.

Optionally, the above-described sulfated and/or phosphated saccharide metal salt matrix containing optical molecules could further contain ferromagnetic and/or superparamagnetic particles. The ferromagnetic and superparamagnetic particles are known to function as MRI contrast agent. Ferromagnetic materials tend to have a strong magnetism; that is, the dipole moments of the atoms tend to spontaneously align in the same direction. This may occur even in the absence of an externally applied magnetic field. Examples of materials that exhibit ferromagnetism include, among others, iron, cobalt, nickel, gadolinium, dysprosium, and their alloys, as well as CrO₂, MnOFe₂O₃, FeOFe₂O₃, NiOFe₂O₃, CuOFe₂O₃, MgOFe₂O₃, EuO, Y₃Fe₅O₁₂, and various iron oxide compounds, such as magnetite (Fe₃O₄) and ferrite (Fe₂O₃). Superparamagnetic materials have a magnetic susceptibility between that of ferromagnetic and paramagnetic materials, and typically are small particles (i.e., below about 150 nanometers). Examples of superparamagnetic materials include various iron containing contrast agents, such as superparamagnetic iron oxides (“SPIOs”), and including ultrasmall SPIOs. Examples of suitable SPIOs include appropriate particle sized magnetite particles (e.g., with a particle size below 10 nm), ferrite particles, maghemite (γ-Fe₂O₃) particles, and magnetoferritin particles. Examples of suitable ultrasmall SPIOs include ferumoxide coated with carboxydextran (Resovist® ferucarbotran, Schering AG), monocrystalline iron oxide nanoparticles coated with Dextran T-10 (Combidex® ferumoxtran, Advanced Magnetics, Inc.), and monocrystalline iron oxide nanopreparations (MION) coated with Dextran T-10 (such as MION-37 having a particle size of 22±6 nm and MION-46 having a particle size of 21±3 nm). Other examples of ferromagnetic and superparamagnetic particles are known in the art and described in U.S. Pat No. 5,653,959, herein incorporated by reference in its entirety, and U.S. Pat. No. 5,023,072. Optionally, the superparamagnetic or ferromagnetic particles could be coated with a suitable material that provides increased stability under acidic conditions. Such materials for coating superparamagnetic or ferromagnetic particles are well known in the art.

In another embodiment, the particles for use in the present invention are optical dye encapsulated in a microsphere particle such as those having a particle size in the range of about 40 μm to about 1200 μm, e.g., trisacryl gelatin microspheres such as those commercially available from BioSphere Medical as Embosphere® microspheres. These microspheres can be physically entrapped within or coated by the sulfated and/or phosphated saccharides of the present invention, particularly the sulfated and/or phosphated saccharide metal salt matrix, and used for the optical imaging applications of the present invention.

Once the optical molecule substituted sulfated and/or phosphated saccharide formulation has been prepared, it may be administered to a patient to aid in the detection of damaged areas of the gastrointestinal tract. As used herein “patient” means any animal in which it may be necessary to detect damaged areas of the gastrointestinal tract. Preferably, the animal is a mammal, and most commonly is a human.

The optical dye substituted sulfated and/or phosphated saccharide compositions described herein can be formulated into diagnostic compositions for administration. When formulated, the optical dye substituted sulfated and/or phosphated saccharide compositions are suitable for in vivo, such as oral or rectal, administration. Preferably the compositions are administered orally. In addition to the optical dye substituted sulfated and/or phosphated saccharide, the compositions may comprise other pharmaceutically acceptable carriers, diluents, or excipients, including buffers, surfactants, emulsifiers, thixotropic agents, stabilizing agents, and flavoring agents and other ingredients for enhancing the organoleptic qualities of the composition. The compositions may also comprise antibiotic or antifingal agents (e.g., paraben) and anti-gas agents (e.g., simethicone). The composition may be formulated into any conventional pharmaceutical administration form, such as tablets, coated tablets, capsules, pills, powders, solutions, suspensions, dispersions, syrups, emulsions, etc.

The dose of the optical molecule substituted sulfated and/or phosphated saccharide containing composition administered is not particularly limited, so long as the dose enables detection of damaged areas of the gastrointestinal tract. The dose will vary depending on the patient, the optical molecule selected, and the detection technique being used, but may readily be optimized by one skilled in the art. In one embodiment, the composition is administered in an amount of about 0.001 g to about 2 g of optical dye substituted sulfated and/or phosphated saccharide per 5 to 50 mL of suspension, preferably the composition is administered in an amount of about 0.001 g to about 1 g of optical dye substituted sulfated and/or phosphated saccharide per 10 to 20 mL of suspension. In another embodiment, the composition is administered in an amount of about 0.001 g to about 2 g of sulfated and/or phosphated saccharide metal salt matrix containing optical molecule per 5 to 50 mL of suspension, preferably the composition is administered in an amount of about 0.001 g to about 1 g of sulfated and/or phosphated saccharide metal salt matrix containing optical molecule per 10 to 20 mL of suspension.

The compositions of the present invention are administered to the patient at a suitable time before the optical detection technique is performed. Such suitable time will be well known to those skilled in the art In one embodiment, the compositions are administered at least 15 minutes before the optical detection technique is performed. In another embodiment, the compositions are administered at least 1 hour before the optical detection technique is performed.

Once the optical dye substituted sulfated and/or phosphated saccharide composition is administered, the active components of the composition will adhere to damaged areas of the gastrointestinal mucosa. These damaged areas may then be detected using various optical detection means, such as light imaging techniques. Light imaging technology takes advantage of either transmitted, scattered, or emitted (fluorescence) photons or a combination of these effects. Typically, light imaging techniques use an illumination source in the ultraviolet, visible, or infrared region of the electromagnetic spectrum. In light imaging, the light, which is transmitted through, scattered by, or reflected (or re-emitted in the case of fluorescence) from the body, is detected and an image is directly or indirectly generated. Numerous examples of optical detection means are known in the art. Preferably the detection means is selected from the group consisting of optical tomographic, endoscopic, photoacoustic, and sonofluorescence applications. These and other light imaging techniques are known in the art and described in, for example, U.S. Pat. No. 6,258,340; U.S. Pat. No. 6,540,981; and WO 00/16810.

The present invention additionally provides a method for detecting damaged areas of the gastrointestinal tract. The method comprises administering to a patient a composition comprising an optical molecule containing, e.g., optical dye substituted, saccharide molecule and detecting damaged areas of the gastrointestinal tract with a detection means. Preferably, the saccharide is selected from the group consisting of sulfated saccharide, phosphated saccharide, and combinations thereof. More preferably, the sulfated saccharide is sucralfate, and the optical dye contains one or more groups selected from —SO₃H, —PO₄H₂, —COOH, or combinations thereof wherein the dye does not contain aliphatic chains or groups substituted with primary amine, secondary amine, aldehyde or ketone groups. Optionally, an MRI contrast agent in an amount sufficient to enable detection by magnetic resonance imaging, i.e. a superparamagnetic or ferromagnetic particle, is physically incorporated into the sulfated and/or phosphated saccharide composition containing the optical molecule. In such a case, magnetic resonance imaging may be used to detect the damaged areas of the gastrointestinal tract, instead of or in addition to optical detection means. It will thus be apparent to those skilled in the art that the detection means employed may vary depending on the optical molecule used.

The use of optical molecules in combination with sulfated and/or phosphated saccharide to detect ulcerated and inflamed sites of the gastrointestinal tract may provide numerous advantages over previously known methods of detection which utilize conventional dyes. For example, the specificity of sucralfate for damaged sites of the gastrointestinal tract allows for specific targeting of ulcers using a lower dose of the optical dye-substituted sulfated and/or phosphated saccharide, as compared to conventional contrast agents which are not ulcer-specific. This in turn increases the ease and accuracy of diagnosis which reduces the risk to the patient. In addition, the use of optical dyes provides a means for non-radioactive detection. Furthermore, since sucralfate is only minimally absorbed by the body, most sucralfate molecules that do not form a coating over the inflamed or ulcerated tissue should simply pass through the gastrointestinal tract. As a result, the relative absorption by the body of dye compounds attached to the sucralfate should be decreased relative to absorption of unattached dye compounds. This would result in a safer composition and reduced background artifacts during detection, which gives improved imaging specificity. The low dose volume, safety of ingredients, and low potential absorption through the gastrointestinal tract should also result in minimal patient side effects, thus resulting in improved patient compliance.

Attachment of an optical dye to sulfated and/or phosphated saccharide molecules, and in particular to sucralfate, according to the present invention, is not expected to result in the problem of fluorescence quenching. In addition, attachment of an optical dye to sulfated and/or phosphated saccharide molecules according to the present invention is not expected to block the optical properties of the dyes.

As various changes could be made in the above described compositions and methods without departing from the scope of the invention, it is intended that all matter contained in the above description be interpreted as illustrative and not in a limiting sense.

1-26. (canceled)

27. A composition for detecting damaged areas of the gastrointestinal tract, the composition comprising:
a molecule or a pharmaceutically acceptable salt thereof, the molecule comprising a saccharide substituted with an optical dye, wherein the saccharide is selected from the group consisting of a sulfated saccharide, a phosphated saccharide, and combinations thereof, and wherein the molecule is capable of selectively binding to damaged and/or inflamed areas of the gastrointestinal tract.

28. The composition of claim 27, wherein the saccharide is selected from the group consisting of sucralfate, precursors of sucralfate, and derivatives of sucralfate.

29. The composition of claim 27, wherein the molecule is suitable for optical detection in the gastrointestinal tract.

30. The composition of claim 27, wherein the optical dye transmits, reflects, or fluoresces light in the wavelength range of about 350 nanometers to about 1300 nanometers, and is sufficiently stable under conditions found in the gastrointestinal tract.

31. The composition of claim 27, wherein the optical dye comprises at least one substituent selected from the group consisting of —SO₃H (sulfonic acid), —PO₄H₂ (phosphoric acid), —COOH (carboxylic acid), and combinations thereof.

32. The composition of claim 27, wherein the optical dye is selected from the group consisting of cyanines, indocyanines, phthalocyanines, squaraines, polymethines, pyrazines, rhodamines, fluoresceins, and derivatives and analogues thereof.

33. The composition of claim 27, wherein the optical dye is selected from the group consisting of cyanines, indocyanines, phthalocyanines, merocyanines, carbocyanines, napthocyanines, rhodamines, tetramethyl rhodamines, phenoxazines, phenothiazines, phenoselenazines, fluoresceins, porphyrins, porphyrin analogs, benzoporphyrins, squaraines, corrins, croconiums, azo compounds, methines, triphenylmethinines, polymethines, indoleniums, styryls, oxonols, squariliums, eosins, erythrosin, coumarin, methyl-coumarins, pyrene stibenes, fullerenes, oxatellurazoles, phenylxanthenes, and derivatives thereof.

34. The composition of claim 27, wherein the optical dye is directly attached to the saccharide.

35. The composition of claim 27, wherein the optical dye is indirectly attached to the saccharide by at least one chelator, at least one linker, or at least one coupling moiety.

36. The composition of claim 27, wherein the optical dye and the saccharide are present in a mole ratio of total optical dye to total saccharide of about 0.001:1 to about 3:1.

37. The composition of claim 27, wherein the molecule has a magnetic resonance imaging agent attached thereto.

38. The composition of claim 37, wherein the magnetic resonance imaging agent is selected from the group consisting of paramagnetic agents, superparamagnetic agents, ferromagnetic agents, and combinations thereof.

39. The composition of claim 27, the molecule being of the following formula, wherein: 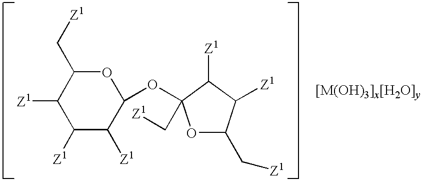
each —Z¹ is independently selected from the group consisting of optical dye, —OR, and any combination thereof, so long as at least one —Z¹ is optical dye;
each —R is independently selected from the group consisting of —SO₃M(OH)₂, —PO₄M(OH)₂, hydrogen, and any combination thereof, so long as at least one —R is —SO₃M(OH)₂ or —PO₄M(OH)₂;
each M is independently selected from the group consisting of Al, Ca, Mg, Na, K, and Ba;
x is 8 to 10; and
y is22to31.

40. The composition of claim 27, the molecule being of the following formula, wherein: 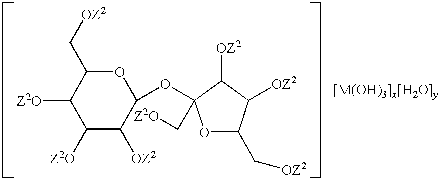
each —Z² is independently selected from the group consisting of optical dyes, —R, hydrogen, and any combination thereof, so long as at least one —Z² is an optical dye;
each —R is independently selected from —SO₃M(OH)₂ and —PO₄M(OH)₂;
each M is independently selected from the group consisting of Al, Ca, Mg, Na, K, and Ba;
x is 8 to 10; and
y is 22 to 31.

41. The composition of claim 27, the molecule being of the following formula, wherein: 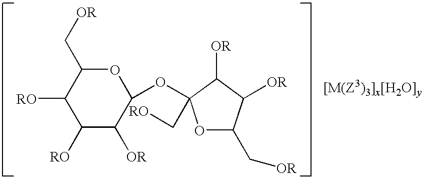
each —R is selected from the group consisting of —SO₃M(Z³)₂, —PO₄M(Z³)₂, hydrogen, and any combination thereof so long as at least one —R is —SO₃M(Z³)₂ or —PO₄M(Z³)₂;
each —Z³ is selected from the group consisting of optical dyes, —OH, and any combination thereof, so long as at least one —Z³ is an optical dye;
each M is independently selected from the group consisting of Al, Ca, Mg, Na, K, and Ba;
x is 8 to 10; and
y is 22 to 31.

42. The composition of claim 27, the molecule being of the following formula, wherein: 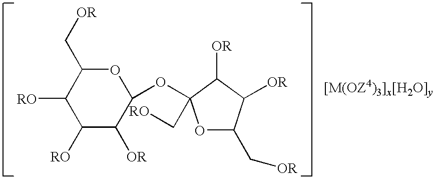
each —R is independently selected from the group consisting of —SO₃M(OZ⁴)₂, —PO₄M(OZ⁴)₂, hydrogen, and any combination thereof so long as at least one —R is —SO₃M(OZ⁴)₂ or —PO₄M(OZ⁴)₂;
each —Z⁴ is selected from the group consisting of optical dyes, hydrogen, and any combination thereof, so long as at least one —Z⁴ is an optical dye;
each M is independently selected from the group consisting of Al, Ca, Mg, Na, K, and Ba;
x is 8 to 10; and
y is 22 to 31.

43. The composition of claim 27, wherein the molecule is a physiologically acceptable particulate material selected from the group consisting of:
nanoparticles suitable for optical imaging; and
optical dye encapsulated in a physiologically acceptable particulate material.

44. The composition of claim 27, wherein the molecule is a nanoparticle suitable for optical imaging selected from the group consisting of:
quantum dots;
nanoparticles of Si or Si/Ge; and
nanoparticles having a suitable optical dye encapsulated therein.

45. The composition of claim 27, wherein the molecule comprises the optical dye encapsulated in a physiologically acceptable particulate material.

46. The composition of claim 45, wherein the physiologically acceptable particulate material is a microsphere.

47. A method of using a composition, the method comprising:
administering to a patient a composition of claim 27, wherein the molecule or pharmaceutically acceptable salt thereof selectively binds to an ulcerated and/or inflamed site of the patient's gastrointestinal tract after being administered; and
detecting the molecule or pharmaceutically acceptable salt thereof in the patient's gastrointestinal tract.

48. The method of claim 47, wherein the detecting is accomplished using at least one of optical tomography, endoscopy, photoacoustic applications, sonofluorescence applications, and magnetic resonance imaging.

49. The method of claim 47, wherein the composition is administered in an amount of about 0.001 g to about 2 g of the molecule or pharmaceutically acceptable salt thereof per about 5 mL to about 50 mL of a suspension.

50. The method of claim 47, wherein the administering occurs at least about 15 minutes prior to the detecting.
